(19) United States
(12) Patent Application Publication
(10) Pub. No.: US 2009/0187067 A1
(43) Pub. Date: Jul. 23, 2009

(54) IMPLANT FOR TREATING EFFORT-RELATED URINARY INCONTINENCE IN WOMEN

(75) Inventors: Patrick CARTERON, Chalain le Comtal (FR); William WIECEK, Bonson (FR)

Correspondence Address: HESLIN ROTHENBERG FARLEY & MESITI PC, 5 COLUMBIA CIRCLE, ALBANY, NY, 12203, US

(73) Assignee: ASPIDE MEDICAL, La Talaudiere (FR)

(21) Appl. No.: 12/356,109

(22) Filed: Jan. 20, 2009

(30) Foreign Application Priority Data
Jan. 22, 2008 (FR) 0850375

(51) Int. Cl. A61F 2/02

(52) U.S. Cl. 600/30

(57) ABSTRACT

An implant for the treatment of urinary incontinence of the type produced as a knitted, woven or other type of strip in polypropylene or similar biocompatible material to be placed under a patient's urethra, has at each end an anchoring area which comprises a deployed structure including one or more fins configured by a succession of folds. The deployed structure being three-dimensional and remembering its shape so that it can be deployed and refolded during the surgical phases of positioning it and fitting and anchoring it through the obturator membrane or, to advantage, in the retropubic space.

The invention falls within the technical sector of permanent implants for treating this type of incontinence. These implants are introduced under the urethra via a vaginal incision and are anchored to the obturator membrane which itself is fixed to the endopelvic surface of the obturator foramen.

These implants are used for the treatment of effort-related urinary incontinence due to urethral hypermobility and/or intrinsic sphincter deficiency, and act as a suburethral hammock.

Various types of implant exist on the market such as those sold under the brands Needleless, from Neodemic, Heletech, from Hesperis Chirurgical, or Miniarc, from AMS. These implants are in the form of very long strips made for example in polypropylene or another biocompatible material with a knitted structure. The ends of these implants are made to be fixed to the obturator membrane.

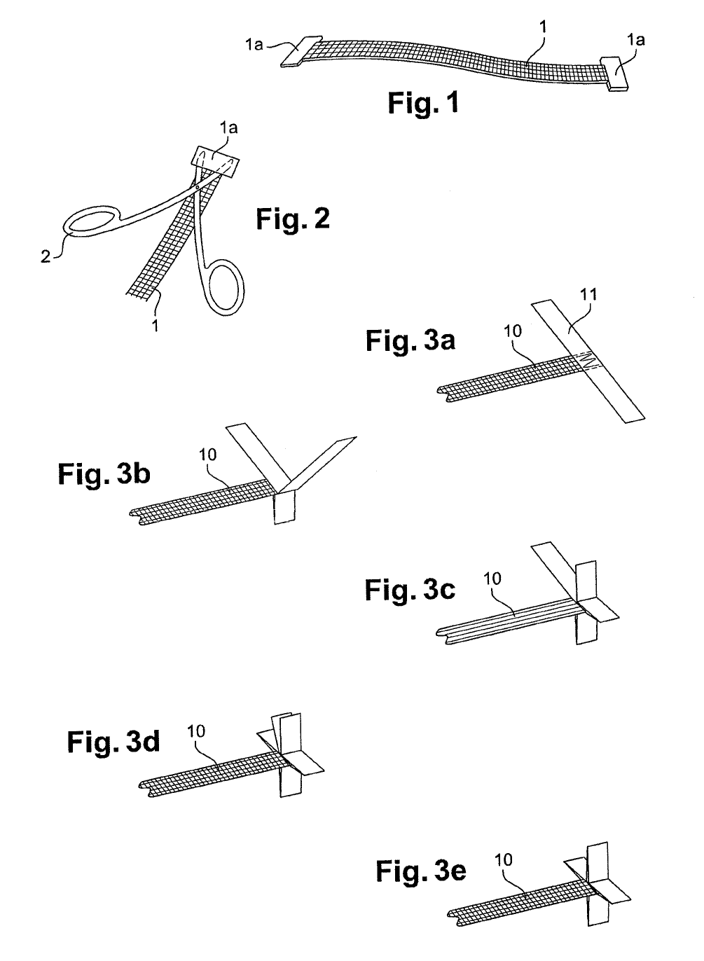

FIGS. 1 and 2 show an example of this type of implant, in particular, the one marketed under the Needleless brand. It consists of a long strip (1) in polypropylene or equivalent material, the ends (1a) of which have wider transverse pockets (1a) into which the instrument (2) for positioning the implant can be temporarily inserted. The wider parts of these pockets, arranged in the same plane as the strip, are used to anchor the implant in the region concerned. One of the problems encountered with this type of strip is its lack of stability, as there is a possibility of the strip slipping relative to the obturator membrane;

Other types of strip have been proposed e.g. in patents EP 1764063 and FR 2859901, in which the ends of the strip are fitted with strengthening in the form of a rigid hollow sleeve to fit on the end of the insertion needle. This type of strip is still complex to make and it does not always remain in place given the configuration of its ends.

Patent WO 2007/059199 gives details of an implant for the treatment of urinary incontinence consisting of a strip placed under the patient's urethra with at its ends a harpoon type of fixation independent of the strip and linked to it by a connecting thread. Positioning remains very uncertain and varies with each fixation end.

The applicant has therefore sought to design a new type of implant based on strips in polypropylene or other compatible material which will remain in place better after fitting and which can be easily implanted by the surgeon.

Another aim was to improve anchorage of the strip through variable arrangements of these anchoring areas, taking into account the morphology of the patient and in particular the area where it is to be fitted.

Another aim was to explore different anatomical regions for anchoring which might be possible because of the improved attachment system.

The applicant's solution meets these objectives while providing an implant which is easy to position with instruments normally used in the field of invasive surgery.

According to a first characteristic of the invention, the implant for the treatment of urinary incontinence, made of a knitted, woven or other form of strip, in polypropylene or similar biocompatible material, for placing below the patient's urethra, at each end of which is an area for fixing to the obturator wall or into the retropubic space, is particular in that at each of its ends the strip has an anchoring area formed by a structure including a number of fins configured by a succession of folds. The said structure when deployed is three-dimensional and remembers its shape so that it can be deployed and refolded in the surgical phases of positioning and fitting it.

According to another characteristic of the invention, the implant is particular in that part of the anchoring structure is in the same plane as that of the strip, and the other part is in a number of planes other than the basic plane defined by the strip, projecting out relative to it thus forming an optimal hooking and anchoring area. This optimal anchoring allows it to be hooked into the retropubic space, avoiding perforation of the obturator membrane, therefore limiting transfixing the latter and other tissues.

These characteristics as well as others will become evident from the rest of the description.

To better understand the object of the invention, it is illustrated, non-exhaustively, through considering the following figures:

FIGS. 1 and 2 are views of an implant according to the state of the art prior to the invention, and relate to the product marketed under the Needleless brand by the company Neodemic, FIG. 1 showing the implant as used and FIG. 2 showing it with use of the fitting instrument.

FIGS. 3a, 3b, 3c, 3d and 3e are views showing how the anchoring area of the implant according to the invention can be shaped, with its various successive phases.

FIG. 4 is a partial view of the implant according to the invention, showing the anchoring area at one end; the other end is not shown but is made in an identical way.

FIG. 5 is a view of the implant according to the invention with a variant form of its anchoring area.

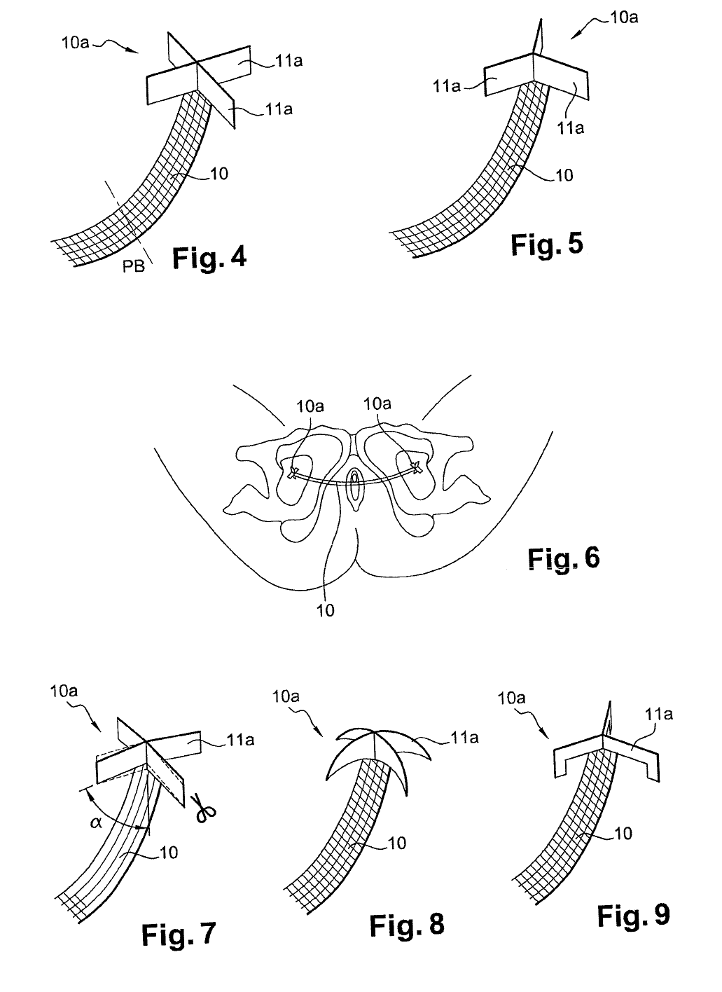

FIG. 6 is a schematic view showing the position of the implant according to the invention, with anchorage to advantage in the retropubic space, or through the obturator membrane.

FIG. 7 shows a variant form of the anchoring area with the cutting diagram for obtaining a harpoon type of anchorage.

FIG. 8 shows the harpoon shape of the fins of the anchoring area.

FIG. 9 shows another configuration of the harpoon type of anchoring area.

To make the object of the invention more concrete, it will now be described in a non-exhaustive manner, illustrated by the figures or drawings.

With reference to FIGS. 4 and 5, the implant for the treatment of urinary incontinence is of the type made as a knitted, plaited, woven or other type of strip (10) in polypropylene or similar biocompatible material to be placed under the patient's urethra. The length of the strip is thus minimal, limiting the quantity of material implanted.

According to the invention, at each of its ends the strip has an anchorage area (10a) consisting of a three-dimensional structure (11) which remembers its shape, so that it can be deployed and refolded in the surgical phases of positioning and fitting it. Part of the anchoring structure (11) is in the same plane as that of the strip, and the other part is in a number of planes other than the basic plane (BP) defined by the strip, projecting out relative to it and thus forming an area for hooking and anchoring to the obturator membrane or, to advantage, in the retropubic space.

This anchoring structure (11) is made in a material identical to or different from that of the strip while being biocompatible.

Each anchoring structure (11) is fixed to one end of the strip by a permanent connection, obtained for example by ultrasonic welding, pressure or other means, to the opposite parts, or by joining with thread or other means.

The anchoring structure is defined by a configuration with several fins (11a), as shown by the examples in FIGS. 4 and 5 with 4 or 3 fins located in different planes from the plane (BP) of the strip. These fins are configured with a knitted mesh identical or different to that of the strip, whilst being of a certain rigidity and firmness to avoid their collapsing onto each other. This finned structure according to the invention is positioned like a harpoon, deployed and anchored on the aforementioned obturator wall or in the retropubic space.

These fins remember their shape which allows them to deploy automatically as shown in FIGS. 4 and 5. There are 3 or 4 or more fins regularly spaced or otherwise through 360°. If necessary, two fins or fewer may be envisaged, but one is in a plane other than the plane (BP) of the strip. Use of an anchoring structure with at least 3 fins is preferred.

The shape of the fins can be rectangular as shown in FIGS. 4 and 5, or curved into a harpoon to provide better anchorage as shown in FIGS. 7, 8 and 9. FIG. 7 shows how the fins are to be cut with scissors or equivalent, after they have been shaped, to give the said fins their final harpoon shape.

Thus according to the invention, and as shown in FIG. 6 illustrating the gynaecological position in the patient, the implant is perfectly held and anchored in the obturator wall in a suburethral position or, to advantage, in the retropubic space.

These implants are fitted using known surgical instruments, the fins being folded back on themselves and not hindering insertion of the implant.

The advantages of the invention are clear. The following points are emphasized:
No need to use resterilisable or disposable ancillary instruments.
The surgical procedure is simple and rapidly performed.
The risks of injury or complications are limited.
Anchoring the implant is improved through the self-redeploying fins for better support of the urethra.
The procedure involves limited dissection and transfixion.

Reference should be made to FIGS. 3a, 3b, 3c, 3d and 3e during explanation of an example of the method for producing the anchoring structure and of how it is shaped at each end of the strip.

These figures show how an anchoring structure is shaped. The same operation is carried out at the other end of the strip.

The strip (10) in FIG. 3a can receive a wider band (11b) at its end which has the characteristics chosen for the finished anchoring structure. This band is deployed and fixed to overlap the end of the strip with parts of it extending on either side of the strip (10). It is affixed to it by any appropriate means, laser, ultrasonic or heat welding, gluing, pressure, the addition of a connecting link or any other means. The band is then shaped with different folds corresponding to the fins to be obtained, each fin being composed of two successive juxtaposed folds. Then, following formation of a fin with two successive folds, it is fixed at the base relative to the part opposite the end of the strip. Thus, to produce the anchoring structure, one fin is defined by being connected to the part of the strip necessary for its fixation and this fin is therefore of necessity in the same plane as the strip, while the other fins formed by successive folds are in other planes creating a three-dimensional anchoring structure. The folds are joined at their base in contact with the strip by ultrasonic welding or other means.

It can therefore be understood from the anchoring structure thus formed that it is held in position through the partial connection of one of the fins with the strip at the place where they overlap and join. A high quality hold is thus guaranteed.

Without departing from the context of the invention and the process for producing the implant, it can be seen that the anchoring structure with several fins can be shaped in advance, and that it can be fixed to the end by any ad hoc means of connection.

The implant for the treatment of urinary incontinence thus designed is easy to fit and provides much better holding characteristics in the obturator wall than earlier types. The implant cannot slip or escape as occurred with the state of the art prior to this invention. With the three-dimensional anchorage a different attachment area can also be used, namely the retropubic space, limiting surgical lesions.

1. Implant for treatment of urinary incontinence comprising a knitted, woven or other type of strip of polypropylene or similar biocompatible material to be placed under a patient's urethra, and having at each end an anchoring area which comprises a deployed structure including one or more fins formed by a succession of folds, the deployed structure being three-dimensional and remembering its shape so that the structure can be deployed and refolded during surgical phases of positioning, and fitting and anchoring the structure through an obturator membrane or in a retropubic space.

2. Implant according to claim 1, wherein a part of the anchoring structure is in a same plane as a plane of the strip, and an other part is in a number of planes other than the plane of the strip, projecting out relative to the plane of the strip thus forming an optimal area for hooking and anchoring onto the obturator membrane.

3. Implant according to claim 1, wherein the anchoring structure is made in an identical or different material from the strip while being biocompatible.

4. Implant according to claim 1, wherein each anchoring structure is fixed at each end of the strip by a permanent connection.

5. Implant according to claim 1, wherein the anchoring structure comprises several fins, certain of the fins being situated in planes other than a plane of the strip.

6. Implant according to claim 1, wherein the anchoring structure is comprised of a number of fins, obtained by shaping an additional band fixed to the strip, and each fin is configured with two adjacent folds, and one of the fins is connected to a part of the strip needed to fix said band, said fin being in a same plane as the strip while the other fins formed by successive folds are in other planes creating a three-dimensional anchoring structure.

7. Process for the production of an implant for treatment of urinary incontinence according to claim 1, comprising: placing at each end of the strip a wider band having characteristics selected for a finished anchoring structure, said band being deployed and fixed by overlapping the end of the strip, and the band then being shaped by different folds corresponding to the fins to be obtained, each fin being composed of two successive juxtaposed folds, and then fixing the fins in the form of folds at a base relative to a part opposite the end of the strip.
